(19) United States
(12) Patent Application Publication
(10) Pub. No.: US 2025/0227194 A1
(43) Pub. Date: Jul. 10, 2025

(54) VIDEO-RECORDING CONTROL DEVICE AND VIDEO RECORDING METHOD

(71) Applicant: JVCKENWOOD Corporation, Yokohama-shi (JP)

(72) Inventors: Kentaro Kodama, Yokohama-shi (JP); Norihiro Yamamoto, Yokohama-shi (JP)

(21) Appl. No.: 19/090,483

(22) Filed: Mar. 26, 2025

Related U.S. Application Data
(63) Continuation of application No. PCT/JP2023/036176, filed on Oct. 4, 2023.

(30) Foreign Application Priority Data
Dec. 2, 2022 (JP) 2022-193758

(51) Int. Cl. H04N 5/77; A61B 5/00; A61B 5/024; G06V 20/59; G06V 40/16; G11B 27/34

(52) U.S. Cl. CPC H04N 5/77; A61B 5/02427; A61B 5/6893; G06V 20/59; G06V 40/176; G11B 27/34

(57) ABSTRACT

A control device includes a video-data acquiring unit that controls a camera used in a vehicle, and that acquires imaging data captured by the camera; a biometric-information detecting unit that detects biometric information of an occupant of the vehicle, and a fluctuation in the biometric information; an imaging-data processing unit that generates an imaging file to which a thumbnail image is associated, from the imaging data; a thumbnail-image generating unit that generates, when the biometric-information detecting unit detects a fluctuation in the biometric information, a thumbnail image from video of a predetermined range including before and after a point of time of detection; and a recording control unit that records the imaging file generated by the imaging-data processing unit and the thumbnail image generated by the thumbnail-image generating unit in a recording unit, associating with each other.

### CROSS-REFERENCE TO RELATED APPLICATIONS

This application is a Continuation of PCT International Application No. PCT/JP2023/036176 filed on Oct. 4, 2023 which claims the benefit of priority from Japanese Patent Application No. 2022-193758 filed on Dec. 2, 2022, the entire contents of both of which are incorporated herein by reference.

### BACKGROUND OF THE INVENTION

### 1. Field of the Invention

The present invention relates to a video-recording control device and a video recording method.

### 2. Description of the Related Art

Drive recorders that primarily capture video of an area outside a vehicle have become widespread for vehicles, and an imaging file is generated and recorded at predetermined intervals of, for example, 60 seconds. Moreover, when an event such as an accident is detected, a video of a predetermined period including the event detection time is stored as event data. For example, in JP-A-2020-141170, a drive recorder that records event data and displays a thumbnail image corresponding to the event data, thereby enabling selection of the event data to be reproduced is disclosed.

Video recording devices such as drive recorders, which continuously generate and record imaging files, can be used to record video of surroundings while a vehicle is running, in addition to video of an accident detected as an event. Such a thumbnail image is generated from an image of the first frame of an imaging file or event data (event file), and is displayed in a list to enable selection of respective files. In such a case, the displayed thumbnail image can fail to reflect the event that is desired to be viewed, and there is a case in which it is difficult to find desired video based on the display of the thumbnail image even when a thumbnail image for each file is displayed.

### SUMMARY OF THE INVENTION

It is an object of the present invention to at least partially solve the problems in the conventional technology.

The above and other objects, features, advantages and technical and industrial significance of this invention will be better understood by reading the following detailed description of presently preferred embodiments of the invention, when considered in connection with the accompanying drawings.

A video-recording control device according to the present disclosure comprising: a video-data acquiring unit that controls a camera used in a vehicle, and that acquires imaging data captured by the camera; a biometric-information detecting unit that detects biometric information of an occupant of the vehicle, and a fluctuation in the biometric information; an imaging-data processing unit that generates an imaging file to which a thumbnail image is associated, from the imaging data; a thumbnail-image generating unit that generates, when the biometric-information detecting unit detects a fluctuation in the biometric information, a thumbnail image from video of a predetermined range including before and after a point of time of detection; and a recording control unit that records the imaging file generated by the imaging-data processing unit and the thumbnail image generated by the thumbnail-image generating unit in a recording unit, associating with each other.

A video recording method that is performed by a video recording device, the method according to the present disclosure comprising: controlling a camera used in a vehicle, and acquiring imaging data captured by the camera; detecting biometric information of an occupant of the vehicle, and a fluctuation in the biometric information; generating an imaging file to which a thumbnail image is associated, from the imaging data; generating, when a fluctuation in the biometric information is detected at the detecting biometric information, a thumbnail image from video of a predetermined range including before and after a point of time of detection; and recording the imaging file generated at the generating an imaging file and the thumbnail image generated at the generating a thumbnail image in a recording unit, associating with each other.

### BRIEF DESCRIPTION OF THE DRAWINGS

FIG. 1 is a block diagram illustrating a configuration example of a video recording device including a control device according to a first embodiment;

FIG. 2 is a diagram explaining an imaging file;

FIG. 3 is a flowchart illustrating an example of a flow of general generation processing of an imaging file in the control device according to the first embodiment;

FIG. 4 is a flowchart illustrating an example of a flow of processing of associating a thumbnail image in the control device according to the first embodiment;

FIG. 5 is a diagram explaining the imaging file and event data; and

FIG. 6 is a flowchart illustrating an example of a flow of processing of associating a thumbnail image in a control device according to a second embodiment.

### DETAILED DESCRIPTION OF THE PREFERRED EMBODIMENTS

Hereinafter, embodiments of a video-recording control device and a video recording method according to the present disclosure will be explained in detail with reference to the accompanying drawings. The following embodiments are not intended to limit the present invention.

### First Embodiment

### Video Recording Device

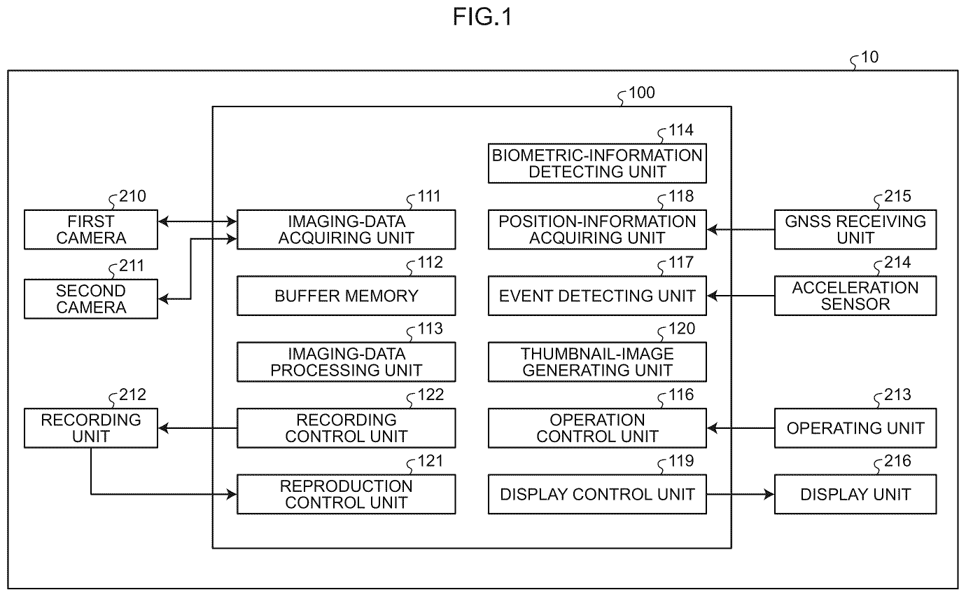

FIG. 1 is a block diagram illustrating a configuration example of a video recording device 10 that includes a video-recording control device (hereinafter, “control device”) 100 according to a first embodiment. The video recording device 10 is a so-called drive recorder that records an event that occurs to a vehicle. The video recording device 10 records an imaging file, associating a thumbnail image therewith.

The video recording device 10 may also be a portable device that can be used in a vehicle, in addition to one mounted on the vehicle. Furthermore, the video recording device 10 may be implemented including a device that is equipped in a vehicle in advance, and a function or a configuration such as a navigation system. The video recording device 10 includes a first camera 210, a second camera 211, a recording unit 212, an operating unit 213, an acceleration sensor 214, a global navigation satellite system (GNSS) receiving unit 215, a display unit 216, and a control device 100. In the present embodiment, the second camera 211 is not an essential component and may be replaced by a sensor capable of detecting biometric information, such as a heart rate, facial expression, or changes in facial expression of an occupant of the vehicle, a portable electronic devices worn by the occupant, or the like.

The first camera 210 is a camera equipped in the vehicle. The first camera 210 is a camera that images surroundings of the vehicle. The first camera 210 may be multiple camera groups. The first camera 210 is positioned, for example, at a position at which a front of the vehicle can be captured, at a front of the interior of the vehicle. In the present embodiment, the first camera 210 continuously images video while an accessory power of the vehicle is ON. The first camera 210 outputs captured imaging data to an imaging-data acquiring unit 111 of the control device 100. The imaging data is a moving image that is constituted of, for example, 27.5 frames per second.

The second camera 211 is a camera equipped in the vehicle. The second camera 211 is a camera for imaging an occupant of the vehicle. The second camera 211 is a camera that performs imaging such that biometric information of the occupant can be detected. The second camera 211 is a camera that enables detection of, for example, a heart rate of the occupant as biometric information. The second camera 211 is a camera that can detect, for example, a facial expression of the occupant as biometric information. The second camera 211 may be multiple camera groups. The second camera 211 is arranged at a position at which an occupant of each seat of the vehicle can be imaged. In the present embodiment, the second camera 211 continuously images video while an accessory power of the vehicle is ON. The second camera 211 outputs captured imaging data to the imaging-data acquiring unit 111 of the control device 100. The imaging data is a moving image that is constituted of, for example, 27.5 frames per second.

The recording unit 212 is used for temporary storage of data in the video recording device 10. The recording unit 212 is, for example, a semiconductor memory device such as a random access memory (RAN) and a flash memory, or a recording unit such as a memory card. Alternatively, it may be an external recording unit that is wirelessly connected through a communication device not illustrated. The recording unit 212 records an imaging file, which is continuous recording data (normal file) or event data.

The operating unit 213 can accept various kinds of operations with respect to the video recording device 10. The operating unit 213 is, for example, a touch panel equipped in the display unit 216. For example, the operating unit 213 can accept an operation of manually storing captured imaging data in the recording unit 212 as event data. For example, the operating unit 213 can accept an operation of reproducing the continuous recording data or event data recorded in the recording unit 212. For example, the operating unit 213 can accept an operation of deleting the event data recorded in the recording unit 212. For example, the operating unit 213 can accept an operation of ending continuous recording. The operating unit 213 outputs operation information to an operation control unit 116 of the control device 100.

The acceleration sensor 214 is a sensor that detects acceleration acting on the vehicle. The acceleration sensor 214 outputs a detection result to an event detecting unit 117 of the control device 100. The acceleration sensor 214 is, for example, a sensor that detects acceleration in three axial directions. The three axial directions include a front-rear direction, a left-right direction, and an up-down direction of the vehicle.

The GNSS receiving unit 215 is constituted of a GNSS receiver that receives a GNSS signal from a GNSS satellite, and the like. The GNSS receiving unit 215 outputs a received position information signal to a position-information acquiring unit 118 of the control device 100.

The display unit 216 is a display device dedicated to the video recording device 10, a display device shared with other systems including a navigation system, or the like. The display unit 216 may be formed integrally with the first camera 210. The display unit 216 is, for example, a display including a liquid crystal display or an organic electroluminescence (EL) display. In the present embodiment, the display unit 216 is arranged in front of the driver of the vehicle, on a dashboard, an instrument panel, or a center console. The display unit 216 displays video based on a video signal output from a display control unit 119 of the control device 100. The display unit 216 displays video being imaged by the first camera 210, or video recorded in the recording unit 212.

### Video-Recording Control Device

The control device 100 is an arithmetic processing device (control device) constituted of, for example, a central processing unit (CPU) or the like. The control device 100 loads a stored program onto a memory, and executes commands included in the program. The control device 100 includes an internal memory not illustrated, and the internal memory is used as a temporary storage of data in the control device 100. The control device 100 includes the imaging-data acquiring unit 111, a buffer memory 112, an imaging-data processing unit 113, a biometric-information detecting unit 114, the operation control unit 116, an event detecting unit 117, a position-information acquiring unit 118, a display control unit 119, a thumbnail-image generating unit 120, a reproduction control unit 121, and a recording control unit 122.

The imaging-data acquiring unit 111 controls the first camera 210 and the second camera 211 used in the vehicle, and acquires imaging data captured by the first camera 210 and the second camera 211. The imaging-data acquiring unit 111 outputs acquired imaging data to the buffer memory 112.

The buffer memory 112 is an internal memory equipped in the control device 100, and is a memory that temporarily records imaging data of a predetermined time period acquired by the imaging-data acquiring unit 111, while updating.

The imaging-data processing unit 113 generates an imaging file per predetermined time from the imaging data. The imaging-data processing unit 113 generates an imaging file to which a thumbnail image is associated, from the imaging data. The imaging-data processing unit 113 converts the imaging data temporarily stored in the buffer memory 112 into, for example, an arbitrary file format, such as an MP4 format, coded by a codec of any format, such as H.264 and moving picture experts group (MPEG)-4. The imaging-data processing unit 113 generates an imaging file as a file of a predetermined time period from the imaging data temporarily stored in the buffer memory 112. As a specific example, the imaging-data processing unit 113 generates an imaging file with imaging data of 60 seconds in order of recording from the imaging data temporarily stored in the buffer memory 112. The imaging-data processing unit 113 outputs the generated imaging file to the recording control unit 122. Moreover, the imaging-data processing unit 113 outputs the generated imaging file to the display control unit 119 after decoding an image through the reproduction control unit 121. A period of the generated imaging file is 60 seconds as an example, but it is not limited thereto. The imaging data herein may be data including audio in addition to video captured by the first camera 210.

The biometric-information detecting unit 114 acquires biometric information in an occupant of the vehicle and a fluctuation of the biometric information. The biometric-information detecting unit 114 detects that the biometric information has become an abnormal stated from a normal state as the variation of the biometric information. In the present embodiment, the biometric-information detecting unit 114 detects that the biometric information has changed from the normal state to the abnormal state during a normal recording period being a period in which normal recording processing is performed.

The biometric-information detecting unit 114 detects a heart rate of the occupant of the vehicle from video of the second camera 211 imaging the occupant of the vehicle, when the biometric information to be detected is heart rate. The biometric-information detecting unit 114 detects a heart rate of the occupant of the vehicle, for example, based on fluctuations in green band wavelength absorbed by hemoglobin in the blood of the face of the occupant of the vehicle or the like captured by the second camera 211. The biometric-information detecting unit 114 can use any other heart rate detection methods. The biometric-information detecting unit 114 detects the heart rate information of the occupant of the vehicle, and detects that the heart rate has risen from the heart rate in a normal state as an abnormal state, as a fluctuation in heart rate. The biometric-information detecting unit 114 detects that it has changed from a normal state to an abnormal state, for example, when the heart rate of the occupant of the vehicle rises to 100 beats per minute or higher within a period of less than five seconds while it is generally detected to be between approximately 60 beats per minute and 70 beats per minute. In the present embodiment, the biometric-information detecting unit 114 detects that the heart rate has risen from the normal state during the normal recording period. The biometric-information detecting unit 114 may be configured to store an average heart rate in the normal state for each occupant of the vehicle, and to detect a change from the normal state to the abnormal state based on fluctuations in heart rate from the average heart rate.

The biometric-information detecting unit 114 detects, when the biometric information to be detected is a facial expression, a facial expression of an occupant of the vehicle from video of the second camera 211 capturing the occupant of the vehicle. The biometric-information detecting unit 114 detects the face of the captured occupant, and detects movement of each part that makes up the face, particularly a degree of eye opening, to detect a fluctuation in facial expression using a facial expression recognition model trained on various facial expressions by machine learning. The biometric-information detecting unit 114 detects a facial expression of an occupant of the vehicle, and detects that the facial expression has changed from the facial expression in a normal state to a facial expression that indicates surprise as the abnormal state, as a fluctuation in facial expression. The biometric-information detecting unit 114 detects, as a fluctuation in facial expression, for example, that a facial expression other than that indicating surprise to a facial expression indicating surprise, particularly, that it has changed within a period of less than one second, as a change from the normal state to the abnormal state. In the present embodiment, the biometric-information detecting unit 114 detects that a facial expression has changed from the facial expression in the normal state to a facial expression that indicates surprise during the normal recording period.

The operation control unit 116 acquires operation information of an operation accepted by the operating unit 213. For example, the operation control unit 116 acquires reproduction operation information indicating a reproduction operation or deletion operation information indicating a deletion operation, and outputs a control signal. For example, the operation control unit 116 acquires end operation information indicating an operation of ending the normal recording, and outputs a control signal.

The event detecting unit 117 detects an event based on acceleration applied to the vehicle. More specifically, the event detecting unit 117 detects an event based on a detection result of the acceleration sensor 214. The event detecting unit 117 detects that an event has occur when acceleration information acquired from the acceleration sensor 214 is equal to or higher than a threshold.

The position-information acquiring unit 118 acquires position information indicating a current position of the vehicle. The position-information acquiring unit 118 calculates the position information of a current position of the vehicle by a publicly-known method based on a GNSS signal received by the GNSS receiving unit 215.

The display control unit 119 controls display in the display unit 216. The display control unit 119 outputs a video signal to be output by the display unit 216, such as the regular recording data or the event data. More specifically, the display control unit 119 outputs the video signal to be displayed by reproduction of video being captured by the first camera 210, or the regular recording data or the event data recorded by the recording unit 212.

The thumbnail-image generating unit 120 generates a thumbnail image from images constituting an imaging file.

The thumbnail-image generating unit 120 adds the thumbnail image to the imaging file. More specifically, the thumbnail-image generating unit 120 normally adds the thumbnail image from an image at the first frame in the video constituting the imaging file.

The thumbnail-image generating unit 120 generates a thumbnail image from video at a point of time of detection when the biometric-information detecting unit 114 detects a fluctuation in the biometric information. More specifically, the thumbnail-image generating unit 120 generates the thumbnail image from the video at the time of detection when the biometric-information detecting unit 114 detects that the biometric information has changed from the normal state to the abnormal state. The video from which the thumbnail-image generating unit 120 generates the thumbnail image may be set for any points of approximately two to five seconds before and after from the point of time when the fluctuation in biometric information is detected.

The thumbnail-image generating unit 120 generates a thumbnail image based on the image at the time of fluctuation of the biometric information when there is a fluctuation in biometric information during the normal recording period. More specifically, the thumbnail-image generating unit 120 adds an image of a frame captured at the time of the fluctuation of biometric information as a thumbnail image of the normal file (regular recording data). The thumbnail image thus generated is highly likely to capture an event around the vehicle that has caused the fluctuation in biometric information of the occupant.

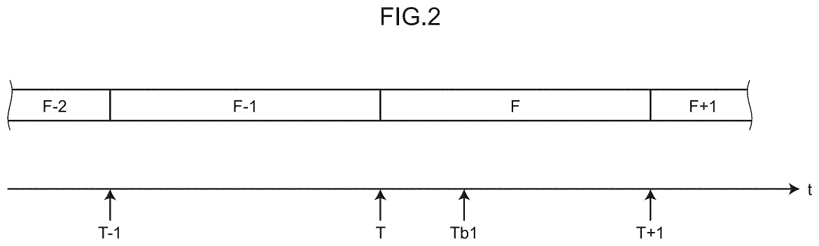

The imaging file as the normal file will be explained using FIG. 2. FIG. 2 is a diagram explaining the imaging file. In the example illustrated in FIG. 2, for example, an imaging file as a normal file per 60 seconds is generated from the imaging data captured by the first camera 210. The normal file is continuously recorded during a period in which the vehicle is in operation, and the normal file is generated and recorded. The regular recording file is an overwritable file.

An imaging file F−1 is generated from imaging data of a period between time T−1 and time T. It is assumed that a fluctuation in biometric information is not detected during the period between time T−1 and time T. In this case, a thumbnail image of the imaging file F−1 is generated from an image at time T−1, in other words, from an image of the first frame of the recording file F−1. The regular recording period of the imaging file F−1 is the period between time T−1 and time T.

The imaging file F is generated from the imaging data of a period between time T and time T+1. It is assumed that a fluctuation of biometric information is detected at time Tb1 within the period between time T and time T+1. In this case, the thumbnail image of the imaging file F is generated from an image at tie Tb1. The regular recording period of the imaging file F is the period between time T and time T+1.

The reproduction control unit 121 controls to reproduce the regular recording data or the event data recorded in the recording unit 212 based on the control signal of a reproduction operation output from the operation control unit 116. The reproduction control unit 121 includes a decoder not illustrated, and decodes provided compressed data, thereby reproducing various kinds of data.

The recording control unit 122 performs recording of imaging data acquired by the imaging-data acquiring unit 111. In the embodiment, the recording control unit 122 performs a control of causing the recording unit 212 to record the imaging file generated by converting into a file by the imaging-data processing unit 113. The recording control unit 122 records the imaging file generated by converting into a file by the imaging-data processing unit 113 in the recording unit 212 as a rewritable imaging file during the period in which the regular recording processing is performed, such as when the accessory power of the vehicle is ON. More specifically, the recording control unit 122 continues to record imaging files generated by the imaging-data processing unit 113 in the recording unit 212 during the period in which the regular recording processing is performed, and overwrites the oldest recording file with a new recording file when the storage of the recording unit 212 reaches full capacity.

The recording control unit 122 stores, when an event is detected by the event detecting unit 117, event data that is imaging data corresponding to detection of the event. The event data is an imaging file during an event recording period, which is a predetermined period in the imaging file generated by the imaging-data processing unit 113. The recording control unit 122 stores the imaging file corresponding to the detection of the event in the recording unit 212 as event data protected from overwriting. The recording control unit 122 copies, when an event is detected by the event detecting unit 117, for example, an imaging file of a predetermined period of approximately 10 seconds before and after a point of time when the event is detected from the buffer memory 112, and stores as the event data.

The recording control unit 122 records the imaging file generated by the imaging-data processing unit 113 and the thumbnail image generated by the thumbnail-image generating unit 120 in the recording unit 212 in an associated manner.

### Information Processing in Video-Recording Control Device

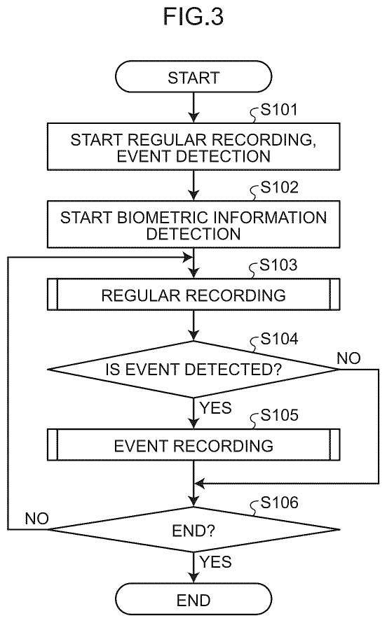

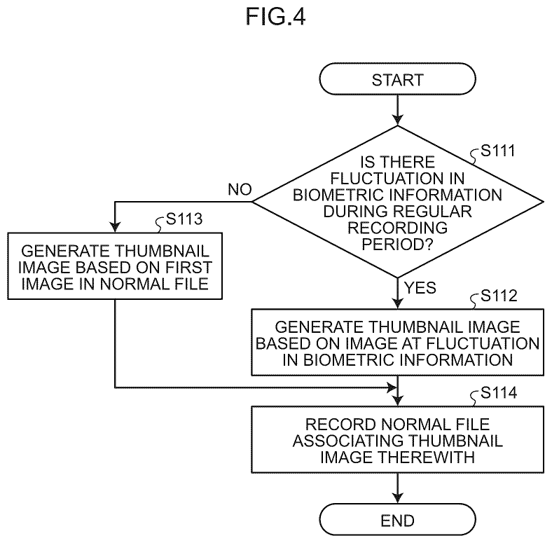

Next, a flow of processing in the control device 100 will be explained using FIG. 3 and FIG. 4. FIG. 3 is a flowchart illustrating an example of a general flow of imaging-file generation processing in the control device 100 according to the first embodiment. FIG. 4 is a flowchart illustrating an example of a flow of processing of associating a thumbnail image in the control device 100 according to the first embodiment.

Recording processing will be explained using FIG. 3. While the video recording device 10 is operating, the processing in the flowchart in FIG. 3 is performed.

With a start of the processing in FIG. 3, the control device 100 starts regular recording and event detection (step S101), and the regular recording by the recording control unit 122 and the event detection by the event detecting unit 117 are continued while the processing in FIG. 3 is performed, in other words, while the video recording device 10 is operating. The control device 100 proceeds to step S102.

With the start of the processing in FIG. 3, the control device 100 starts detection of biometric information (step S102), and continues the detection of biometric information while the processing in FIG. 3 is performed, in other words, while the video recording device 10 is operating. More specifically, the control device 100 acquires biometric information of an occupant of the vehicle and a fluctuation in biometric information by the biometric-information detecting unit 114. More specifically, the control device 100 detects an increase in heart rate from the normal state as an abnormal state by the biometric-information detecting unit 114. The control device 100 detects, for example, a change from a normal expression to an expression that indicates surprise as an abnormal state, as a fluctuation in a facial expression by the biometric-information detecting unit 114. The control device 100 proceeds to step S103.

The control device 100 continues the regular recording processing while the processing in FIG. 3 is performed (step S103). The control device 100 proceeds to step S104.

The control device 100 determines whether an event has been detected (step S104). More specifically, the control device 100 detects that an event has been detected by the event detecting unit 117 when the acceleration information acquired from the acceleration sensor 214 is equal to or higher than a threshold. When it is determined that an event has been detected (step S104: YES), the control device 100 proceeds to step S105. When it is not determined that an event is detected (step S104: NO), the control device 100 proceeds to step S106.

When it is determined that an event has been detected (step S104: YES), the control device 100 performs event recording processing (step S105). The control device 100 proceeds to step S106.

In the event recording processing at step S105, the control device 100 causes the recording control unit 122 to store captured video data as the event data in the recording unit 212, prohibiting from overwriting. The event data is image data that includes time before and after the point of time of event detection.

When it is determined that an event is not detected (step S104: NO), the control device 100 proceeds to step S106 while continuing the regular recording processing.

In the regular recording processing, the control device 100 causes the recording unit 212 to store captured image data as a rewritable normal file by the recording control unit 122.

The control device 100 determines whether to end the processing (step S106). For example, it is determined to end the processing based on that the power or propulsion of the vehicle is OFF, or that the operating unit 213 is operated. When it is determined to end the processing (step S106: YES), the control device 100 ends this processing. When it is not determined to end the processing (step S106: NO), the control device 100 proceeds to step S103. In other words, the regular recording, the event detection, and the biometric information detection are continued.

Next, processing of adding a thumbnail image to a normal file when the regular recording processing indicated at step S103 in FIG. 3, that is, the regular recording is performed will be explained using FIG. 4. Processing in the flowchart in FIG. 4 is performed in the regular recording processing.

The control device 100 determines whether there is a fluctuation in biometric information during the regular recording period (step S111). More specifically, the control device 100 determines whether the biometric information has changed from the normal state to the abnormal state as a fluctuation in biometric information in the regular recording period, based on a detection result of the biometric-information detecting unit 114. More specifically, the control device 100 determines whether a heart rate has risen from the heart rate in the normal state to be the abnormal state during the regular recording period by the biometric-information detecting unit 114. The control device 100 determines whether a facial expression has changed from a normal expression to an expression that indicates surprise as an abnormal state, as a fluctuation in a facial expression during the regular recording period, by the biometric-information detecting unit 114. When it is determined that there is a fluctuation in biometric information during the regular recording period (step S111: YES), the control device 100 proceeds to step S112. When it is not determined that there is a fluctuation in biometric information during the regular recording period (step S111: NO), the control device 100 proceeds to step S113.

When it is determined that there is a fluctuation in biometric information during the regular recording period (step S111: YES), the control device 100 generates a thumbnail image based on an image at the time of the fluctuation in biometric information (step S112). More specifically, the control device 100 adds an image of a frame captured at the time of fluctuation in biometric information as the thumbnail image of that normal file, by the thumbnail-image generating unit 120. The control device 100 proceeds to step S114. Applying to the example in FIG. 2, a fluctuation in biometric information is detected at time Tb1 within the regular recording period (between time T and time T+1) in which the imaging file F being a normal file is generated. Therefore, the image of the frame captured at time Tb1 is added as the thumbnail image of the imaging file F.

When it is not determined that there is a fluctuation in biometric information during the regular recording period (step S111: NO), the control device 100 generates a thumbnail image based on the first image in the normal file (step S113). More specifically, the control device 100 adds an image of the first frame in the imaging file as the thumbnail image of the normal file by the thumbnail-image generating unit 120. The control device 100 proceeds to step S114. Applying to the example in FIG. 2, a fluctuation in biometric information is not detected during the regular recording period (between time T−1 and time T) in which the imaging file F−1 being a normal file is generated. Therefore, an image of the first frame in the imaging file F−1 captured at time T−1 is added as the thumbnail image of the imaging file F−1.

The control device 100 records the normal file, associating the thumbnail therewith (step S114). More specifically, the control device 100 records the imaging file as a normal file generated by the imaging-data processing unit 113 and the thumbnail image generated by the thumbnail-image generating unit 120, associating with each other in the recording unit 212 by the recording control unit 122.

As described, the control device 100 generally records files, associating a thumbnail image from an image of the first frame in video constituting an imaging file. When there is a fluctuation in biometric information during the regular recording period, the control device 100 records files, adding a thumbnail image that is based on an image at the time of fluctuation in biometric information as its thumbnail image.

### Effect

As described above, in the present embodiment, when a fluctuation in biometric information is detected, recording is possible, adding an image at the time of detection of the fluctuation as a thumbnail image. In the present embodiment, when a fluctuation is detected during the regular recording period, recording is possible, adding a thumbnail image based on the image at the time of fluctuation in biometric information. According to the present embodiment, recording is possible, adding a thumbnail image in which an event around a vehicle being a cause of a fluctuation in biometric information of an occupant is highly likely captured. According to the present embodiment, it is possible to facilitate search for recorded normal files.

In the present embodiment, when it is detected that the biometric information has changed from the normal state to the abnormal state, a thumbnail image can be generated from video at the time of detection.

In the present embodiment, when a heart rate has risen from the heart rate in the normal during the regular recording period, a thumbnail image based on an image at the time of fluctuation in biometric information can be added to be recorded.

In the present embodiment, when a facial expression changes from an expression in the normal state to an expression that indicates surprise during the regular recording period, a thumbnail image based on an image at the time of fluctuation in biometric information can be added to be recorded.

### Second Embodiment

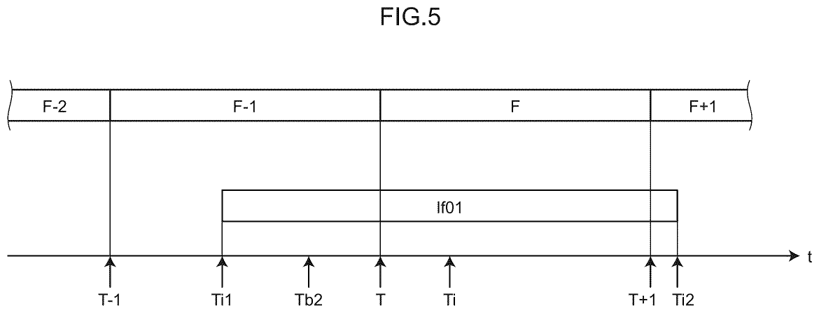

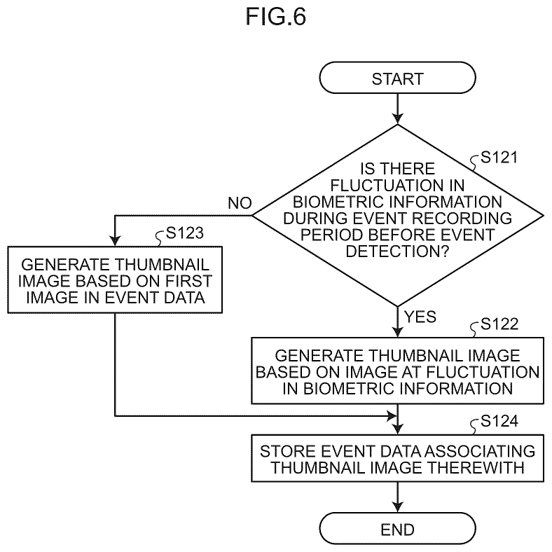

The video recording device 10 according to the present embodiment will be explained, referring to FIG. 5 and FIG. 6. FIG. 5 is a diagram explaining an imaging file and event data. FIG. 6 is a flowchart illustrating an example of a flow of processing of associating a thumbnail image in the control device 100 according to a second embodiment. A basic configuration of the video recording device 10 is same as that of the video recording device 10 according to the first embodiment. In the following explanation, identical reference symbols or corresponding reference symbols are assigned to components similar to those of the video recording device 10, and detailed explanation thereof will be omitted. In the present embodiment, processing in the biometric-information detecting unit 114 is different from that in the first embodiment.

In the present embodiment, the biometric-information detecting unit 114 detects that the biometric information has changed from the normal state to the abnormal state during event recording period in which event recording processing is performed before event detection.

In the present embodiment, the biometric-information detecting unit 114 detects that a heart rate has risen from the heart rate in the normal state in the event recording period before event detection, when biometric information to be detected is a heart rate.

In the present embodiment, the biometric-information detecting unit 114 detects that a facial expression in the normal state has changed to an expression that indicates surprise during the event recording period before event detection, when biometric information to be detected is a facial expression.

The thumbnail-image generating unit 120 adds a thumbnail image to an imaging file, which is event data. More specifically, the thumbnail-image generating unit 120 normally adds the thumbnail image from an image of the first frame in video constituting the imaging file, which is the event data.

When there is a fluctuation in biometric information during the event recording period before event detection, the thumbnail-image generating unit 120 generates a thumb nail image based on an image at the time of fluctuation in biometric information. More specifically, the thumbnail-image generating unit 120 adds an image of a frame captured at the time of the fluctuation in biometric information as a thumbnail image of the event data.

The event data and the imaging file will be explained using FIG. 5. The imaging file F−1, the imaging file F, and an imaging file F+1 are similar to those in the first embodiment. For the event data, imaging data of predetermined period before and after the point of time when the event is detected is stored as event data prohibited from overwriting.

For example, when an event is detected at time Ti, imaging data of a period between time Ti1 and time Ti2 is stored as event data If01. When an event is detected at time Ti, for example, if a fluctuation in biometric information is not detected during the period between time Ti1 and time Ti, the thumbnail image of the event data If01 is generated from an image of the first frame in the event data If01, in other words, from an image at time Ti1. When an event is detected at time Ti, and when a fluctuation in biometric information is detected at time Tb2 within the period between time Ti1 and time Ti, the thumbnail image of the event data If01 is generated from an image at time Tb2, not the image at time Ti1. The event recording period before event detection of the event data IF01 is the period between time Ti1 and time Ti.

Next, event recording processing indicated at step S105 in FIG. 3, that is, processing of adding a thumbnail image to event data when event recording is performed will be explained using FIG. 6. In processing at step S122, processing similar to that at step S112 in the flowchart illustrated in FIG. 4 is performed. Processing in the flowchart in FIG. 6 is performed in the event recording processing.

The control device 100 determines whether there is a fluctuation in biometric information during the event recording period before event detection (step S121). More specifically, the control device 100 determines whether the biometric information has changed from the normal state to the abnormal state as a fluctuation in biometric information during the event recording period before event detection, based on a detection result of the biometric-information detecting unit 114. More specifically, the control device 100 determines whether a heart rate has risen from the heart rate in the normal state to be the abnormal state during the event recording period before event detection by the biometric-information detecting unit 114. The control device 100 determines whether a facial expression has changed from a normal expression to an expression that indicates surprise as an abnormal state, as a fluctuation in a facial expression during the event recording period before event detection, by the biometric-information detecting unit 114. When it is determined that there is a fluctuation in biometric information during the event recording period before event detection (step S121: YES), the control device 100 proceeds to step S122. When it is not determined that there is a fluctuation in biometric information during the event recording period before event detection (step S121: NO), the control device 100 proceeds to step S123.

When it is not determined that there is a fluctuation in biometric information during the event recording period before event detection (step S121: NO), the control device 100 generates a thumbnail image based on the first image in the event file (step S123). More specifically, the control device 100 adds an image of the first frame in the event data as the thumbnail image of the event data by the thumbnail-image generating unit 120. The control device 100 proceeds to step S124. Applying to the example in FIG. 5, when a fluctuation in biometric information is not detected during the event recording period before event detection (between time Ti1 and time Ti) in the event data If01, an image of the first frame in the event data If01 captured at time Ti1 is added as the thumbnail image of the event data If01.

Applying the processing at step S122 to the example in FIG. 5, a fluctuation in biometric information is detected at time Tb2 within the event recording period before event detection (between time Ti1 and time Ti) in the event data If01. Therefore, the image of the frame captured at time Tb2 is added as the thumbnail image of the event data If01.

The control device 100 stores event data, associating the thumbnail image therewith (step S124). More specifically, the control device 100 records the imaging file as the event data generated by the imaging-data processing unit 113 and the thumbnail image generated by the thumbnail-image generating unit 120, associating with each other in the recording unit 212, by the recording control unit 122.

As described, the control device 100 normally adds a thumbnail image from an image of the first frame in video constituting an imaging file, to be recorded. When there is a fluctuation in biometric information during the event recording period before event detection, the control device 100 adds a thumbnail image that is based on an image at the time of fluctuation in biometric information to be recorded.

### Effect

As described above, according to the present embodiment, it is possible to facilitate search for captured event data.

The video recording device according to the present disclosure may be implemented in various different forms other than the embodiments described above.

The respective components of the video recording device illustrated are of functional concept, and it is not necessarily required to be configured physically as illustrated. That is, specific forms of the respective devices are not limited to the ones illustrated, and all or some thereof can be configured to be distributed or integrated functionally or physically in arbitrary units according to various kinds of loads, usage conditions, and the like.

The configuration of the video recording device is implemented, for example, by a program loaded to a memory as software, and the like. In the embodiments described above, it has been explained as a functional block implemented by cooperation of these hardware and software. That is, these functional blocks can be implemented in various forms with hardware only, software only, or a combination of those.

The component described above include those that can be easily conceived by a person skilled in the art and those that are substantially identical. Furthermore, the described configurations can be combined as appropriate. Moreover, various omissions, substitutions, or modifications of the configuration can also be made within a range not departing from the gist of the present invention.

In the above description, it has been explained as including the second camera 211, but detection of a heart rate of an occupant of a vehicle is not limited thereto. The detection of a heart rate is not limited to detection by video captured by the second camera 211, but also various methods by various kinds of sensors embedded in a seat of the vehicle, a smartwatch, and the like can also be applied.

According to the present disclosure, an effect of facilitating search for captured video is produced.

Although the invention has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. A video-recording control device comprising:
a video-data acquiring unit that controls a camera used in a vehicle, and that acquires imaging data captured by the camera;
a biometric-information detecting unit that detects biometric information of an occupant of the vehicle, and a fluctuation in the biometric information;
an imaging-data processing unit that generates an imaging file to which a thumbnail image is associated, from the imaging data;
a thumbnail-image generating unit that generates, when the biometric-information detecting unit detects a fluctuation in the biometric information, a thumbnail image from video of a predetermined range including before and after a point of time of detection; and
a recording control unit that records the imaging file generated by the imaging-data processing unit and the thumbnail image generated by the thumbnail-image generating unit in a recording unit, associating with each other.

2. The video-recording control device according to claim 1, wherein
the biometric-information detecting unit detects that the biometric information has changed from a normal state to an abnormal state, as a fluctuation in the biometric information, and
the thumbnail-image generating unit generates, when the biometric-information detecting unit detects that the biometric information has changed from the normal state to the abnormal state, a thumbnail image from video at the time of detection.

3. The video-recording control device according to claim 2, wherein
the biometric-information detecting unit detects heart rate information of the occupant of the vehicle, and detects that a heart rate has risen from the heart rate in a normal state, as the abnormal state

4. The video-recording control device according to claim 2, wherein
the biometric-information detecting unit detects a facial expression of the occupant of the vehicle, and that detects that the facial expression has changed to a facial expression indicating surprise, as the abnormal state.

5. The video-recording control device according to claim 1, wherein
the imaging-data processing unit generates an imaging file per predetermined time from the imaging data, and
the thumbnail-image generating unit generates, when a fluctuation in the biometric information is detected during a recording period of the imaging file, generates a thumbnail image of the imaging file from video of a predetermined range including a point of time of detection.

6. The video-recording control device according to claim 1, further comprising
an event detecting unit that detects an event based on acceleration applied to the vehicle, wherein
the recording control unit stores, when an event is detected by the event detecting unit, event data that is imaging data corresponding to detection of the event, and
the thumbnail-image generating unit generates, when a fluctuation in the biometric information is detected during a period before event detection in the event data, a thumbnail image of the event data from video of a predetermined range including a point of time of detection.

7. A video recording method that is performed by a video recording device, the method comprising:
controlling a camera used in a vehicle, and acquiring imaging data captured by the camera;
detecting biometric information of an occupant of the vehicle, and a fluctuation in the biometric information;
generating an imaging file to which a thumbnail image is associated, from the imaging data;
generating, when a fluctuation in the biometric information is detected at the detecting biometric information, a thumbnail image from video of a predetermined range including before and after a point of time of detection; and
recording the imaging file generated at the generating an imaging file and the thumbnail image generated at the generating a thumbnail image in a recording unit, associating with each other.
